(19) United States
(12) Patent Application Publication
(10) Pub. No.: US 2010/0286705 A1
(43) Pub. Date: Nov. 11, 2010

(54) VASCULAR ACCESS TO EXTRA-VASCULAR SPACE

(75) Inventor: Thomas A. Vassiliades, JR., Atlanta, GA (US)

Correspondence Address: McKeon Meunier Carlin & Curfman LLC, 817 W. Peachtree Street, Suite 900, Atlanta, GA, 30308, US

(73) Assignee: EMORY UNIVERSITY, Atlanta, GA (US)

(21) Appl. No.: 12/811,892

(22) PCT Filed: Jan. 30, 2009
(86) PCT No.: PCT/US09/32679

§ 371 Date: Jul. 7, 2010

Related U.S. Application Data
(60) Provisional application No. 61/024,958, filed on Jan. 31, 2008.

(51) Int. Cl. A61B 17/00

(52) U.S. Cl. 606/108

(57) ABSTRACT

Provided are methods for accessing tissue in the vicinity of a blood vessel. Also provided are devices for use in a lumen of a patient's organ and methods for shunting interior volumes of organs.

### CROSS REFERENCE TO RELATED APPLICATIONS

This application claims the benefit of U.S. Ser. No. 61/024,958, filed Jan. 31, 2008, which is incorporated herein by reference in its entirety.

### FIELD OF THE INVENTION

This invention pertains to methods and apparatus for accessing the anatomical or potential space or structures of a patient exterior of a blood vessel or organ having a lumen. For example, this invention pertains to such methods and apparatus for accessing such space or structures from the patient's blood vessel while maintaining blood flow through the vessel.

### BACKGROUND

In cardiovascular surgery and interventional cardiology, there is an ongoing developmental effort to devise new procedures and apparatus to permit minimally invasive therapeutic procedures by using a patient's blood vessels to access a treatment site. For example, it may be desirable to access tissue near a blood vessel for treatment; specifically, the treatment of a tumor by advancing an ablation tool through the patient's blood vessels to a location near the tumor and then passing the ablation tool through the wall of the blood vessel into the tumor for ablation treatment. Another example is accessing a neural or muscular structure near a blood vessel to pass a stimulation electrode through the blood vessel to the structure to be stimulated.

From time to time, it is desirable to connect blood vessels or other biologic conduits or organs in order to create an artificial passageway between the structures using natural or synthetic graft materials. U.S. Pat. No. 6,485,513 to Fan dated Nov. 26, 2002 shows (in FIGS. 7a and 7b), a shunt between blood vessels 20 where the graft is percutaneously delivered for the purpose of performing a vascular bypass. A minimally invasive bypass shunt is also shown in U.S. Pat. No. 6,325,776 to Anderson et al dated Dec. 4, 2001. An intraluminally directed vascular anastomosis of a graft vessel and related apparatus are shown in U.S. Pat. No. 7,220,268 to Blatter dated May 22, 2007. Connection of a conduit with a target vessel is shown in U.S. Patent Application Publication No. U.S. 2004/0097988 to Gittings et al published May 20, 2004 and U.S. Patent Application Publication No. U.S. 2006/0282106 to Cole et al published Dec. 14, 2006.

In addition, the vasculature system can provide a pathway for access to adjacent organs. Such is shown in U.S. Pat. No. 5,755,682 to Knudson et al dated May 26, 1998; U.S. Pat. No. 7,179,270 to Makower dated Feb. 20, 2007 and U.S. Pat. No. 7,159,592 to Makower et al dated Jan. 9, 2007.

### SUMMARY

According to an embodiment of the present invention, a method and apparatus are shown for accessing tissue in the vicinity of a blood vessel by advancing a conduit through the blood vessel to a target site. A distal opening of the conduit is positioned against to and facing a wall of the blood vessel at the target site. A procedure tool is advanced through the conduit to the distal end to form an opening through the wall of the blood vessel to the target site. According to additional embodiments of the present invention, apparatus and methods are shown for shunting interior volumes of two organs such as blood vessels. A first conduit with an open distal end is placed within the lumen of a first blood vessel with the open distal end opposing and sealed against the first vessel's wall. Similarly, a second conduit with an open distal end is placed within the lumen of a second blood vessel with an open distal end opposing and sealed against the second vessel's wall. The first conduit is everted out of the first vessel's lumen and passed into the interior of the second conduit. A graft is passed into the everted first conduit with its ends secured to both the first and second vessels.

### BRIEF DESCRIPTION OF THE DRAWINGS

FIG. 1 is a side cross-sectional schematic of a blood vessel;

FIG. 2 is the view of FIG. 1 showing a flexible sheath introduced into the blood vessel lumen at a distant site and with an open distal end of the sheath sealed to the vessel wall;

FIG. 3 is a bottom plan view of FIG. 2;

FIG. 4 is a view of FIG. 2 with a procedure tool advanced through the sheath;

FIG. 5 is a bottom plan view of FIG. 4;

FIG. 5a is a side cross-sectional schematic of a blood vessel;

FIG. 6 is the view of FIG. 2 showing a stent within the blood vessel and with one embodiment of a sheath within the stent for guiding a treatment tool to a target site;

FIG. 7 is the view of FIG. 6 with a second embodiment of a sheath within the stent and having a catheter within the sheath for guiding a tool to the treatment site;

FIG. 8 is a still further embodiment of a sheath within the stent and showing an alternative design of a catheter for guiding a tool to a target site;

FIG. 9 is a perspective view of an expanded stent with a sheath according to the present invention;

FIG. 10 is a side sectional view of the stent and sheath of FIG. 9;

FIG. 11 is a bottom plan view of the stent of FIG. 9;

FIG. 12 is a side sectional view of the stent and sheath of FIGS. 9-11 placed within a blood vessel;

FIG. 13 is the view of FIG. 12 showing an inflatable balloon in lieu of a stent;

FIG. 14 is the view of FIG. 12 showing the sheath everted through and out of the blood vessel and with a free end of the sheath tied off;

FIG. 15 is the view of FIG. 14 in combination with a side sectional view of a second blood vessel and with a graft material introduced into the everted sheath of FIG. 14 and with a second sheath placed within the second blood vessel;

FIG. 16 is the view of FIG. 15 with the everted sheath and graft passed into the second sheath of the second blood vessel;

FIG. 17 is a completed view of a graft placed between two blood vessels according to the present invention;

FIG. 18 is an enlarged view of a portion of FIG. 16;

FIG. 19 is an enlarged view of a portion of FIG. 17;

FIG. 20 is a side sectional view of an alternative embodiment according to the present invention and showing an inflatable balloon with a center tube and a flexible sheath;

FIGS. 21 and 22 show alternative embodiments of the balloon of FIG. 20 in a deflated state; and

FIG. 23 shows the balloon of FIG. 20 in an inflated state;

FIGS. 24a-f are schematic illustrations of an alternative embodiment according to the present invention.

### DETAILED DESCRIPTION

Referring now to the several drawing figures in which identical elements are numbered identically throughout, a description of example methods and devices will now be presented. In the various drawing figures, the present invention is illustrated in embodiments where the apparatus of the present invention is placed within a blood vessel for access to tissue or organs exterior to the blood vessel. It will be appreciated that the present invention can also be applied to placement of the apparatus in any hollow body organ for access to tissue or organs exterior to the organ in which the apparatus is placed.

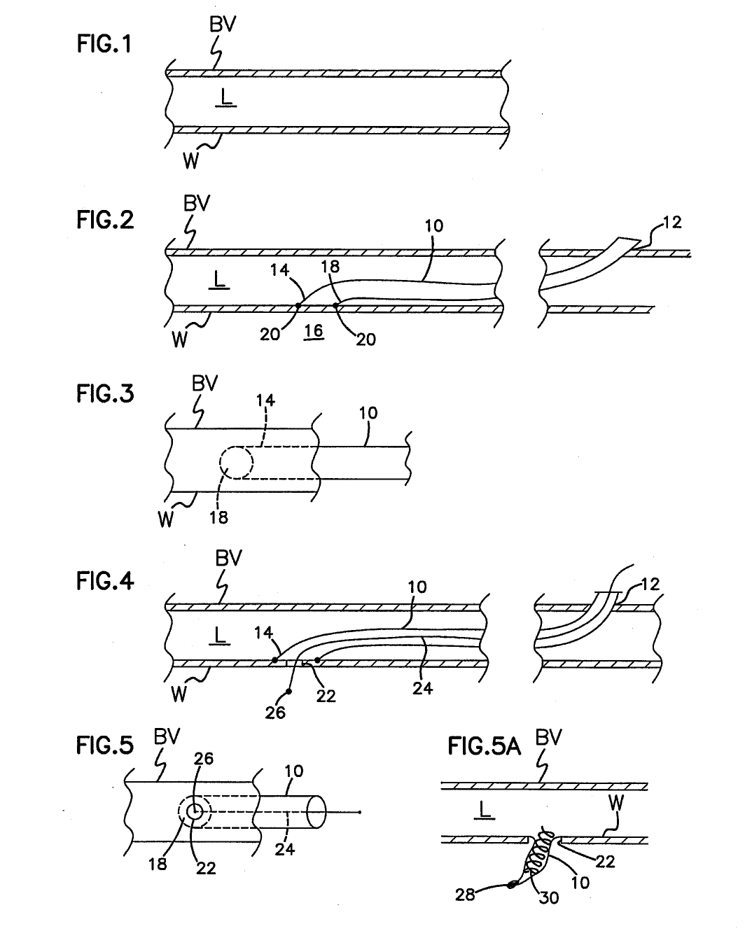

With initial reference to FIGS. 1-5, a blood vessel BV is shown having a vessel wall W defining an interior lumen L. FIG. 2 shows a flexible sheath 10 according to the present invention. A conduit, such as the sheath 10, is introduced through the side wall W of the blood vessel BV at a distant site 12. For example, the sheath 10 could be introduced into the blood vessel (or a connecting blood vessel) in the leg of the patient and the sheath distal end 14 advanced to a target site 16. Thus, a conduit sized to pass through a lumen is sized to be advanced within the lumen to a target site in the lumen for accessing a tissue target site. The target site 16 may be tissue external to the blood vessel BV to be treated or may be a location near an adjacent blood vessel or other organ to which a graft is to be connected.

The distal end 14 has an opening 18 which is sealed to the side wall W of the blood vessel BV by any suitable means as schematically illustrated at 20. Example mechanisms for sealing the distal end 14 against the side wall W are disclosed later in this application but can include other techniques for sealing such as those disclosed in aforementioned patent application Publication Nos. U.S. 2006/0282106 and U.S. 2004/00997988, both incorporated herein by reference as though set forth in full.

With the distal end 14 sealed to the side wall W, a hole (for example hole 22 in FIG. 4) can be formed through the side wall W by means of any incision tool passed through the flexible conduit 10. As a consequence of the seal 20, blood flow is maintained through the lumen L and does not leak through the hole 22.

FIG. 4 illustrates a treatment tool schematically at 24. The treatment tool 24 is admitted to the sheath 10 external to the body and passed through the sheath 10 and through the hole 22 while at all times maintaining blood flow in the lumen L.

The treatment tool 24 is shown schematically as having a treatment tip 26. The treatment tip 26 may be an ablation tip for ablating tissue. An example would be a tip for admitting RF energy, laser energy for cryogenic therapy. Also, the tip 26 may be an electrode for providing stimulation therapy. The treatment tool 24 may be left in place following removal of the sheath 10 (for example in the case of a stimulating electrode) or the treatment tool 24 may be removed from the sheath 10 following treatment. The treatment tool can be used to access a number of normal or diseased tissues. For example, the tool can be used to ablation of a tumor, or for accessing a neural or muscular structure to stimulate the structure. Therapeutic agents, such as anti-cancer drugs, can also be delivered using the tool.

FIG. 5a illustrates the sheath being tied off at 28 and the sheath 10 everted through the hole 22 so that the sheath 10 is completely exterior to the lumen L after completion of a therapy procedure at a target site. If desired, a filling substance 29 can be placed within the everted sheath 10 to prevent blood pooling within the sheath 10. Such filling substances 29 are well known in the treatment of aneurysms and include embolic coils and the like as disclosed in U.S. Pat. No. 6,179,857. Alternatively, the sheath 10 can be severed at seal 20 and removed and hole 22 repaired.

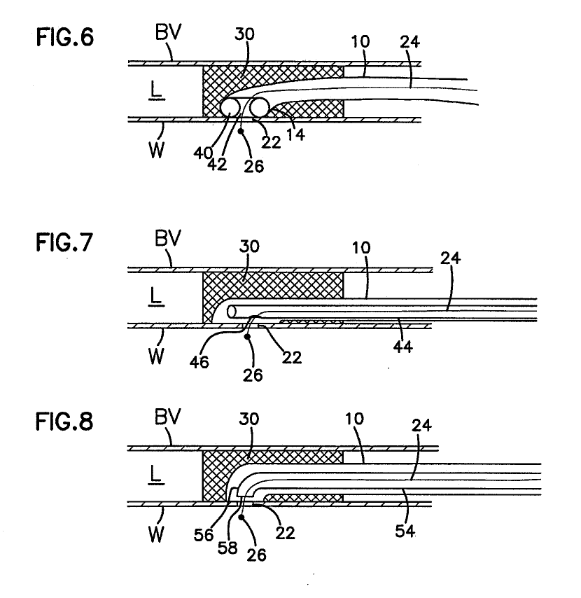

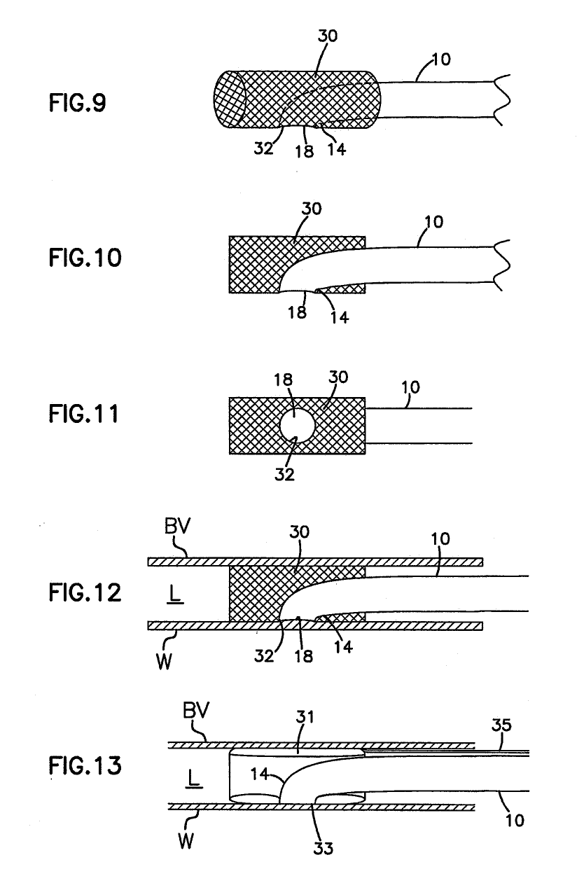

FIGS. 6-12 illustrate alternative embodiments for sealing an opening 18 of a distal end 14 of a sheath 10 against the wall W of a blood vessel BV. Each of the embodiments of FIGS. 6-8 includes an expandable stent 30 containing a flexible sheath 10. In all of the embodiments shown, the stent 30 is shown as a wire mesh expandable stent and illustrated in an expanded state. It will be appreciated that there are a very large number of expandable stent designs and one of which could be applicable for use in the present invention. The stent 30 may be self-expanding or balloon expanding. The stent can be delivered in any conventional manner for stent delivery.

The stent 30 is generally cylindrical in configuration and has a side hole 32 through its cylindrical wall. The sheath 10 has its distal end 14 secured to the wall of the stent with the distal opening 18 of the sheath 10 aligned with the opening 32 of the stent.

When the stent 30 is expanded within a blood vessel BV (FIG. 12), the expansion of the stent 30 urges the distal end 14 of the sheath 10 against the wall W of the blood vessel in sealing engagement so that a hole can be formed through the wall W at the opening 18 without leakage of blood from the lumen L. Blood flow is permitted to continue through the lumen L and through the hollow expandable stent 30. With the embodiment of FIG. 12, a tool (such as tool 24 in FIG. 4) can be passed through the sheath 10 to form a hole 22 through the side wall W at distal opening 18. Blood does not leak out of the lumen L through hole 22.

FIGS. 6-8 illustrate alternative embodiments to accurately position such a tool 24 centrally within the opening 18 of the sheath 10. In FIG. 6, an expandable toroidal balloon 40 is positioned at the distal end 14 of the sheath 10. Expansion of the balloon 40 following placement of the stent 30 defines a balloon center 42 through which the tool 24 can be passed with assurance that the tool 24 is aligned in the center of the distal opening 18.

In FIG. 7, a semi-rigid catheter 44 is passed through the sheath 10. At its distal end, the catheter 44 has an opening 46 in its side wall to aim the tool 24 through sheath opening 18 to the desired location of hole 22. FIG. 8 is similar to that of FIG. 7 except that the catheter 54 has an elbow-shaped distal end 56 with an opening 58 opposing the desired location of hole 22.

FIG. 13 shows an alternative to an expandable stent 30 where the sealing mechanism is an expandable balloon 31 having a side opening 33 to which the distal end 14 of sheath 10 is secured. An inflation line 35 is provided for inflating and deflating the balloon 31. In all the embodiments thus described, a procedure can be performed by placing the expanding member 30, 31 within the blood vessel BV at a target site and expanding the expanding member 30, 31 to urge the distal end 14 against the wall W of the blood vessel BV with distal opening 18 opposing a target site. Such action seals the distal end 14 against the wall W of the blood vessel BV. Accordingly, a procedure tool 24 can be advanced through the sheath 10 to form a hole 22 through the wall W. The sealing of the sheath end 14 against the wall W ensures blood flow can be maintained through the lumen L and the expanding member 30, 31 without leakage of the blood flow through the hole 22 formed by the procedural tool 24.

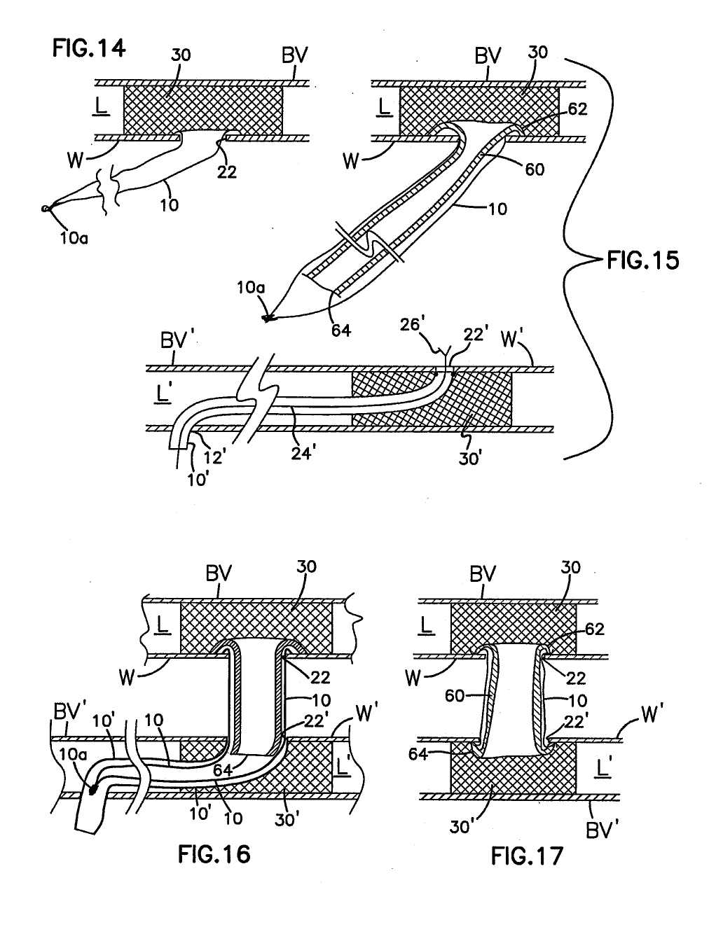

FIGS. 14-17 illustrate the use of the present invention in establishing a shunt or graft between two vessels or organs. FIG. 14 illustrates a sleeve 10 placed in a blood vessel BV as previously described and with the sheath 10 everted outside of the blood vessel and terminating at a tied-off end 10a. FIG. 15 illustrates the first blood vessel BV as previously shown in FIG. 14 with the addition of a graft material 60 positioned within the sheath 10. The graft material 60 can be delivered via a catheter used to previously deliver stent 30.

A first end 62 of the graft 60 is secured to the wall W of the blood vessel BV by any suitable means as previously described or otherwise known to one of ordinary skill in the art. It will be appreciated that throughout the placement of the graft 60 within the sheath 10 and securing the end 62 to the wall W, blood flow is maintained throughout the lumen L without leakage of the blood flow through the hole 22 in the wall W. The graft material 60 can be any suitable graft material such as a blood vessel harvested from the patient or a synthetic graft material.

FIG. 15 also illustrates an adjacent blood vessel BV' which has received a stent 30′ and sheath 10′ delivered in the same manner as previously described with reference to FIG. 12. The apparatus 30′, 10′ and anatomy of the second blood vessel BV' is identical to that of blood vessel BV and apparatus 30, 10 and distinguished by an apostrophe.

A second procedure tool 24′ is schematically shown as being passed through the sleeve 10′ and through the opening 22′. The tool 24′ can be any tool for grabbing the end 10a of the everted sheath 10. The end 10a can be radiopaque to permit identification and capture or could be magnetic and attracted to the distal end 26′ of the grabbing tool 24′.

With the end 10a grabbed by tool 26′, the end 10a is pulled within the second sheath 12′ as illustrated in FIG. 16. This draws the second end 64 of the graft material into the second lumen L′ where the end 64 may then be secured to the wall W′. When secured to the wall the graft can be directly secured to the wall or secured to the wall with a portion of the sheath interposed between the wall and the graft. After such procedure, excess length both of sheath 10 and 10′ may be severed through any suitable means and removed leaving the completed graft in the patient. FIG. 17 illustrates a completed shunt between blood vessel BV and BV'. At all times throughout the procedure, blood flow was maintained in both of lumens L and L′ without leakage through either of hole 22, 22′. Upon completion, blood flow through the graft 60 occurs without substantial contact of blood with any surface other than natural blood vessel BV, BV' or the graft 60.

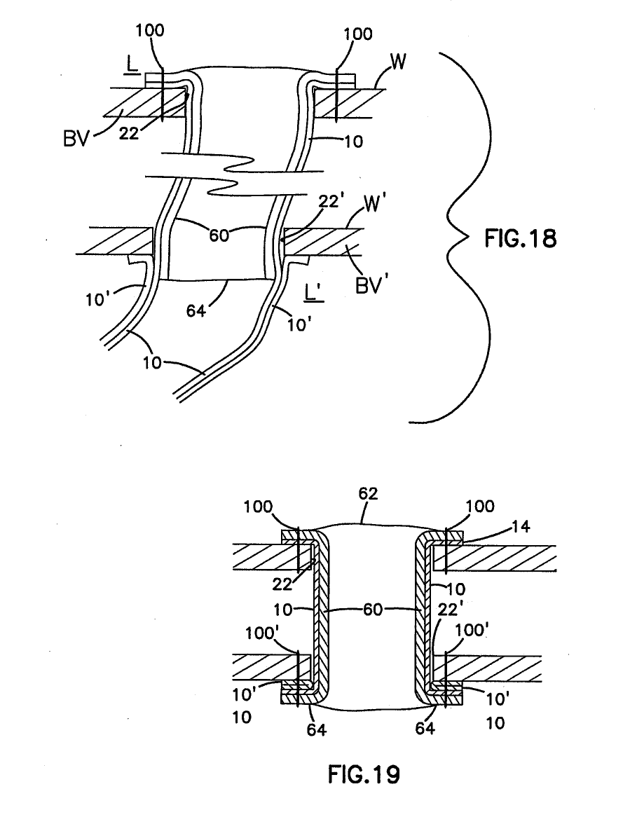

FIGS. 18 and 19 are enlarged views of portions of FIGS. 15 and 17. FIGS. 18 and 19 better show the various layers of material. For ease of illustration, all layers are shown secured to the wall W, W′ by sutures 100, 100′ and illustrate that blood flow is predominantly or exclusively in contact with the graft material or the natural walls W, W′.

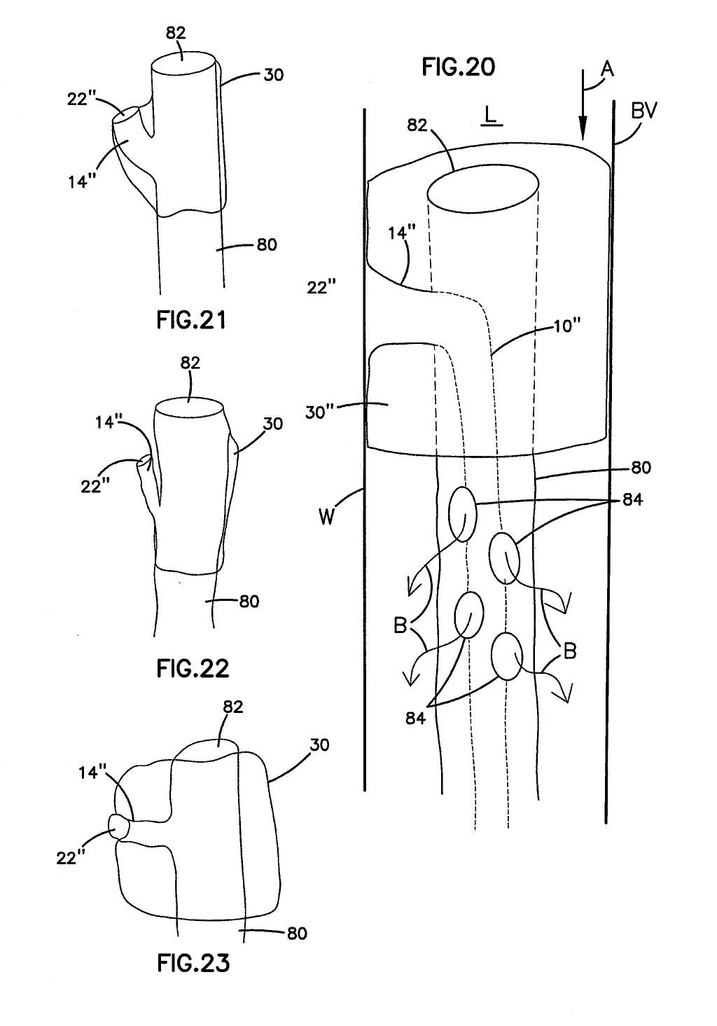

FIG. 22 illustrates an alternative embodiment where the securing apparatus 30″ is an inflatable balloon placed within the blood vessel BV. Centrally positioned within the inflatable balloon is a catheter 80 having an open distal end 82 facing a normal direction of blood flow illustrated by the arrow A. Contained within the catheter 80 is the sheath 10″ with distal end 14″ exposed to a target area 22″ through which the previously described procedures can be performed. Distal to the balloon 30″ are a plurality of holes 84 formed through the side wall of the catheter so that blood flow admitted to the catheter through opening 82 may flow out of holes 84 as illustrated by arrows B. FIGS. 20, 21 show the balloon 30 in a deflated state and FIG. 22 shows the balloon in an inflated state.

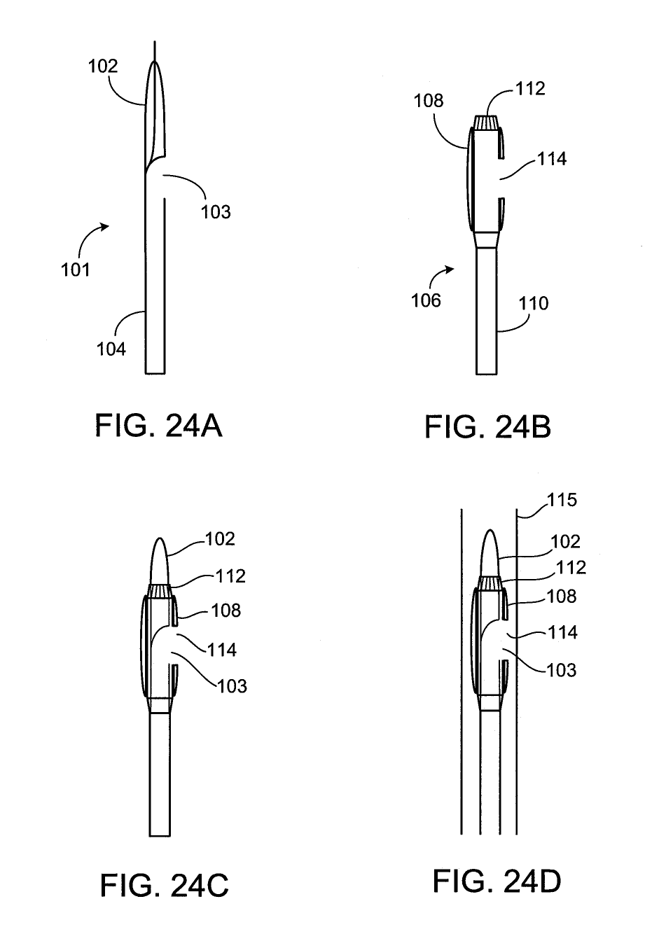

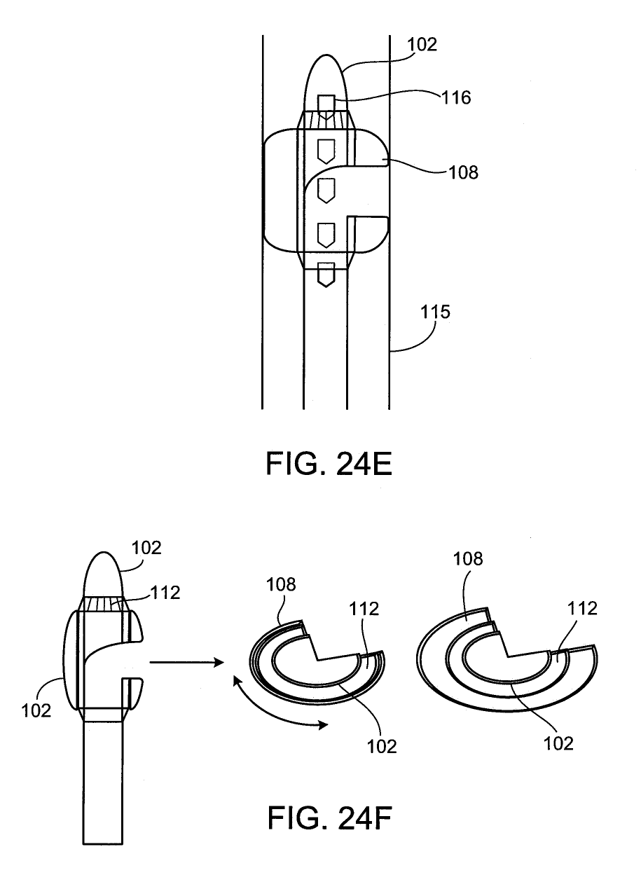

FIGS. 24a-f are schematic diagrams illustrating an alternative embodiment where an expandable member 108 is used to seal the opening 103 of a conduit 101 against a lumen defining wall of an organ. For example, the expandable member can comprise an inflatable balloon.

FIG. 24a shows a conduit 101, for example a catheter. The catheter can comprise an opening 103 at its distal end. For example, the opening 103 can be located on a side of the catheter spaced from the distal extremity or tip of the catheter. The tip 102 of the catheter can be solid and can be configured to allow guide wire insertion through the tip 102 and along a side channel located in the shaft 104 of the catheter. The catheter can be advanced into and through a vessel lumen 115 or organ having a lumen in a subject. A second conduit 106 comprising an expandable member 108 and a shaft 110 can be advanced into and through a vessel lumen 115 or organ having a lumen in a subject in a position overlying or surrounding the outer surface of the conduit 101. The expandable member 108 can comprise a opening 114 that can be aligned with the opening 103 of the conduit 101 as shown in FIG. 24c. The conduit 101 can be rotated relative to the conduit 106 such that the openings 103 and 114 are not aligned and rotated into a position where the openings are aligned.

FIG. 24d illustrates the conduit 101 and second conduit 106 positioned over the conduit 101 and located in the lumen 115 of a vessel, wherein the tip 102 points in the direction opposite of blood flow in the vessel lumen 115. The opening 103 and the opening 114 are aligned. In FIG. 24d the expandable member (e.g. a inflatable balloon) is deflated to an extent to allow for advancement through the vessel lumen 115 so that the openings can be aligned with a target portion of the inner luminal wall of the vessel. In this deflated configuration, blood flow can pass around the device including around the inflatable expandable member. As shown in FIG. 24e, the expandable member 108 can be expanded, for example by inflation, to seal (e.g. secure) the opening 114 against the luminal wall. In this expanded configuration, blood is not able to freely flow around the device because the expandable inflatable member approximates the luminal walls. However, blood flow 116 is maintained through the vessel by flowing though a porous portion 112 of the conduit 106. For example, the porous portion 112 can be positioned between the expandable member 108 and the conduit shaft 104 and tip 102. Although in the expanded configuration blood flow 116 is maintained through the blood vessel, the conduit 101 and wall of the lumen define a space sealed from the blood flow. In this way, an opening can be made in the lumen wall at the portion of the lumen wall that defines the space without having blood flow out of the vessel thought the opening in the lumen wall and while blood flow is maintained through the blood vessel lumen.

The porous portion 112 can have an outer layer and an inner layer wherein blood can flow between the outer layer and inner layer. Optionally, spokes can be positioned between the inner and outer layers to maintain one or more open passages for blood flow even when the expandable inflatable member 108 is inflated and sealed against the luminal walls of the vessel. FIG. 24f illustrates cross sections of the conduit 101 and 106 taken at the level of the openings 103 and 114. The cross section on the left is with the expandable inflatable member 108 in a deflated configuration to allow for passage within a vessel lumen. The cross section on the right is with the expandable inflatable member 108 in an expanded configuration for approximating the luminal walls to seal the opening 114 against a luminal wall. In this configuration a space is defined by the wall portion and the conduit 101. Portions of the space can also be defined by the expandable member 108. The porous portion 112 allows blood to flow along the vessel in both configurations.

The methods and devices can be used to create bypass conduits or communications between cavities, hollow organs or vessels from within the cavities, hollow organs or vessels. For example, the methods and devices can be used to create vascular communications for peripheral artery bypass to treat vascular occlusive disease or to create an arterio-venous fistula for dialysis access. The methods and devices can also be used for bypassing coronary arteries to treat coronary atherosclerosis. The methods and devices can also be used outside of the cardiovascular system. For example, the methods and devices can be used in the digestive or urological system and the natural body orifices can be the external access points rather than peripheral blood vessels.

Through the foregoing detailed description of the present invention it has been shown how the objects of the invention have been attained in the preferred manner. Modifications and equivalents of the disclosed concepts are included within the scope of the claims appended hereto. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed.

What is claimed is:

1. A method for accessing tissue in the vicinity of a blood vessel, the method comprising:
selecting a conduit sized to pass through a lumen of the blood vessel and having an opening at the distal end of the conduit;
placing the distal end of the conduit in the blood vessel lumen and advancing the distal end to a target site with the opening opposing a portion of a wall of the blood vessel at the target site such that a space is sealed from blood flow within the blood vessel lumen; and
advancing a tool though the conduit and through the portion of the blood vessel wall to access tissue in the vicinity of the blood vessel with the tool.

2. The method of claim 1, wherein the conduit and wall portion at least partially define the space sealed from blood flow within the blood vessel lumen.

3. The method of claim 2, wherein the conduit, wall portion, and a portion of an expandable member having an opening aligned with the opening at the distal end of the conduit define the space sealed from blood flow within the vessel lumen.

4. The method of claim 1, wherein the opening at the distal end is spaced from the distal extremity of the conduit.

5. The method of claim 1, wherein the conduit is a flexible sheath.

6. The method of claim 1, further comprising advancing a catheter having an opening at its distal end through the conduit, wherein the tool is advanced through the catheter located in the conduit, the opening at the distal end of the catheter, and the portion of the blood vessel wall to access the tissue in the vicinity of the blood vessel.

7. The method of claim 1, wherein the tool is configured to ablate tissue in the vicinity of the blood vessel.

8. The method of claim 1, wherein the tool comprises a tip for administering one or more of RF energy, laser energy, and cryogenic therapy to the tissue in the vicinity of the blood vessel.

9. The method of claim 1, wherein the tool is configured to deliver one or more therapeutic agents to the tissue in the vicinity of the blood vessel.

10. The method of claim 1, wherein the tool is configured to provide electrical stimulation therapy to the tissue in the vicinity of the blood vessel.

11. The method of claim 1, further comprising removing the conduit from the blood vessel while leaving the tool in its advanced position to access the tissue in the vicinity of the blood vessel subsequent to removal of the conduit.

12. A method for shunting interior volumes of two organs, the method comprising:
placing a first conduit with an opening at its distal end into a first interior volume of a first organ such that the opening opposes a portion of a wall of the first organ;
forming a first hole through the wall portion of the first organ;
placing a second conduit with an opening at its distal end into a second interior volume of a second organ such that the opening opposes a portion of a wall of the second organ;
forming a second hole through the wall portion of the second organ;
everting a portion of the first conduit out of the first interior volume through the first hole;
passing a graft into the everted first conduit from the first interior volume and securing a first end of the graft to the wall of the first organ;
passing a second end of the graft and everted first conduit through the second hole and into the second interior volume and securing the second end of the graft to the wall of the second organ; and
removing first and second conduit material from the second interior volume to create a shunt between the interior volumes of the first and second organs.

13. The method of claim 12, wherein a portion of the first conduit is removed.

14. The method of claim 12, wherein the first organ is a blood vessel.

15. The method of claim 14, wherein the second organ is a blood vessel.

16. The method of claim 12, wherein the graft comprises a blood vessel.

17. The method of claim 12, wherein the graft comprises synthetic graft material.

18. A method for accessing tissue in the vicinity of a blood vessel, the method comprising:
selecting a conduit sized to pass through a lumen of said blood vessel and having an opening at a distal end;
placing the distal in the blood vessel and advancing the distal end to a target site with the opening opposing a portion of a wall of the blood vessel;
securing the distal end against the wall;
forming a opening through the portion of the wall of the blood vessel;
advancing a tool through the conduit and out of the opening through the portion of the wall of the blood vessel.

19. An apparatus for use in a lumen of a patient's organ, comprising:
a conduit sized to pass through the lumen and having an opening at a distal end of the conduit;
an expandable member sized to be placed within the lumen and configured for urging the opening at the distal end into sealing engagement against a lumen-defining wall of the organ to seal the opening from the lumen.

20. The apparatus of claim 19, wherein the expandable member is a stent having a reduced size selected to pass through the lumen and an expanded size such that an exterior surface of the stent is urged against the wall of the organ so that the distal end opening of the conduit is aligned with an opening through the stent.

21. The apparatus of claim 19, wherein the expandable member comprises an inflatable balloon having an inflated configuration for an exterior surface of the balloon to be urged against the wall of the organ such that the opening of the conduit is sealed from the lumen.

22. The apparatus of claim 21, wherein the expandable member further comprises an opening configured to oppose the lumen-defining wall of the organ and wherein the conduit opening is aligned with the opening of the expandable member.

23. The apparatus of claim 22, wherein the lumen has fluid flowing there through and wherein the apparatus is further configured to maintain fluid flow though the lumen when the inflatable balloon is in its inflated configuration.

24. The apparatus of claim 23, wherein the organ is a blood vessel.

25. The apparatus of claim 22, wherein the lumen has fluid flowing there through and wherein the apparatus further comprises a second conduit configured to maintain fluid flow through the lumen when the inflatable balloon in its inflatable configuration.

26. The apparatus of claim 25, wherein the organ is blood vessel.
